(19) United States
(12) Patent Application Publication
(10) Pub. No.: US 2024/0382699 A1
(43) Pub. Date: Nov. 21, 2024

(54) DETERMINING DATA RELATED TO THE APPROACH OF END OF LIFE OF A DRUG DELIVERY DEVICE

(71) Applicant: SANOFI, Paris (FR)

(72) Inventors: Hester Jane Corne, Warwick (GB); Jonathan Jason Drake, Warwick (GB); Paul Richard Draper, Warwick (GB); Ronald Antony Smith, Salir (PT)

(21) Appl. No.: 18/693,855

(22) PCT Filed: Sep. 22, 2022
(86) PCT No.: PCT/EP2022/076289

§ 371 Date: Mar. 20, 2024

(30) Foreign Application Priority Data
Sep. 24, 2021 (EP) 21315186.3

(51) Int. Cl. A61M 5/50; A61M 5/315

(52) U.S. Cl. CPC A61M 5/5086; A61M 5/31568; A61M 2205/3306; A61M 2205/3327; A61M 2205/581; A61M 2205/582; A61M 2205/583; A61M 2205/587; A61M 2205/8206

(57) ABSTRACT

An electronic system (700) being configured and a method for determining data related to the approach of end of life of a drug delivery device (1) are disclosed, wherein the determining of data related to the approach of end of life of the drug delivery device is based one or more of the following: an evaluation of the voltage of an internal battery (29) of the drug delivery device (1) or a drug delivery add-on device; a detection of an error of the drug delivery device (1) or drug delivery add-on device, wherein the error is related to end of life of the drug delivery device (1); an evaluation of a storage capacity of a memory (24) for records of doses expelled with the drug delivery device (1).

### FIELD

The present disclosure relates to determining data related to the approach of end of life of a drug delivery device.

### BACKGROUND

U.S. Pat. No. 8,556,847 B2 and U.S. Pat. No. 7,749,186 B2 relate to devices for delivering substances and methods of making and using them, and more particularly, relate to medical devices for administering or delivering a product in doses, such as injection apparatus, syringes, injection pens, etc., using which a dosed amount of a fluid product, such as insulin, growth hormones or osteoporosis preparations, etc., can be self-administered by a user. An injection device comprises at least one sensor for detecting an operating process of the injection device, an electronic circuit connected to the sensor for establishing the beginning and elapsed time of a service life, based on one or more sensor signals, and an output device connected to the circuit for providing a signal indicating the end of the service life. A method for determining a service life of an injection device is also encompassed, wherein the beginning of the service life is established by one or more sensors for detecting an operating process of the device, a signal is generated which signals the end of the service life, and at least one of an optical, acoustic or tactile output device is associated with the injection device for providing a signal which indicates the end of the service life. A timer can be coupled to the electronic circuit for detecting one of the service life or operating time of the injection device. The signal indicating to the user the end of the service life can be outputted continuously or for a predetermined time period after the at least one sensor detects an attempted operating process.

### SUMMARY

This disclosure describes an electronic system and a method for determining data related to the approach of end of life of a drug delivery device. The determined data can be evaluated for indicating to a user of the drug delivery device the approach of end of life of the drug delivery device.

In an aspect the present disclosure provides an electronic system configured for determining data related to the approach of end of life of a drug delivery device based one or more of the following: an evaluation of the voltage of an internal battery of the drug delivery device or a drug delivery add-on device; a detection of an error of the drug delivery device or drug delivery add-on device, wherein the error is related to end of life of the drug delivery device; an evaluation of a storage capacity of a memory for records of doses expelled with the drug delivery device. The determination of data based on the above allows to increase reliability of the determination of the approach of end of life of the drug delivery device. For example, the characteristics of internal batteries used in injection pens with regard to their voltage over the device's lifetime may allow to improve the accuracy of the determination of end of life of the drug delivery device. But also, some errors of the drug delivery device such as an injection pen, which can be caused for example when a supply voltage from a battery is no longer so stable as at the beginning of the life cycle of the drug delivery device, may be detected for obtaining end of life approach related data. A yet further characteristic of particularly injection pens is the storage capacity of a dose records memory, which may be limited. Thus, the storage capacity, which may decrease over the drug delivery device lifetime, can be also evaluated to obtain drug delivery device end of life related data.

In a further aspect the present disclosure provides a computer-implemented method for determining data related to the approach of end of life of a drug delivery device based one or more of the following: evaluating the voltage of an internal battery of the drug delivery device or a drug delivery add-on device; detecting an error of the drug delivery device or drug delivery add-on device, wherein the error is related to end of life of the drug delivery device; evaluating a storage capacity of a memory for records of doses expelled with the drug delivery device. The method may be for example implemented as part of a firmware of an electronic system implemented in a drug delivery device, which comprises a processor, particularly a microcontroller. The processor can for example execute the method frequently after usage of the drug delivery device, for example after a drug dose has been expelled, upon a user interaction, for example when a user enters a command in a user interface of the drug delivery device or drug delivery add-on device, when data are transmitted from the electronic system to an external device, particularly an external computing device such as a mobile computer (smartphone, tablet computer, handheld computer, laptop computer etc.), and/or with a predetermined period, for example every week or month without any user interaction.

In embodiments, the evaluation of the voltage of an internal battery of the drug delivery device or drug delivery add-on device may comprise a measurement of the voltage of the internal battery and an outputting of the measured voltage. For example, the characteristics of the typical batteries used in drug delivery devices such as injection pens show a typical discharge behaviour over the lifetime, which can be evaluated to obtain data related to the approach of end of life of the drug delivery device.

In embodiments, the evaluation of the voltage of an internal battery of the drug delivery device or drug delivery add-on device may comprise a measurement of the voltage of the internal battery and a setting of a flag if the measured voltage is below a first threshold. As the first threshold a voltage may be selected, which indicates that the battery is moving towards end of life, particularly that the battery is leaving a “plateau” region of its output voltage indicating that it may be soon exhausted. The setting of the flag may be part of a dose record and stored in an internal non-volatile memory of the drug delivery device or drug delivery add-on device with every dose record. So, a flag history may be evaluated from the stored dose records indicating the approach of end of life of the battery and, thus, the drug delivery device using the battery. The flag history may be advantageous when the battery recovers after usage or the temperature slightly increases such that later dose records are not “flagged”. Based on the flag history, an accurate evaluation of the end of life approach of the battery can be obtained.

In embodiments, the evaluation of the voltage of an internal battery of the drug delivery device or drug delivery add-on device may comprise a measurement of the voltage of the internal battery and a storing of an error code in a non-volatile memory if the measured voltage is below a second threshold. A voltage level can be selected as the second threshold, which is slightly above the minimum voltage required by components of the electronic system to normally work, for example the minimum voltage level required by a processor or microcontroller to operate. The error code is then stored permanently in the non-volatile memory so that it may be retrieved later.

In embodiments, the detection of an error of the drug delivery device or drug delivery add-on device may comprise a measurement of a supply voltage of a processor and a performing of a reset of the processor if the measured supply voltage is below a third threshold, wherein the performed processor reset is stored in a non-volatile memory as detected error.

In embodiments, the detection of an error of the drug delivery device or drug delivery add-on device may comprise a measurement of a supply voltage of a processor and a stopping of operation of the processor as long as the measured supply voltage is below a fourth threshold, wherein the processor operation stopping is stored in a non-volatile memory as detected error.

In embodiments, the detection of an error of the drug delivery device or drug delivery add-on device may comprise an evaluation of one or more readings of a sensor of the drug delivery device or drug delivery add-on device and a detection of an error if the evaluation of the one or more readings indicates a low supply voltage.

In embodiments, the evaluation of a storage capacity of a memory for records of doses expelled with the drug delivery device may comprise a determination of the number of dose records expelled with the drug delivery device and currently stored in a pre-allocated storage area of the memory, and a determination of the remaining storage capacity based on the determined number and a maximum number of dose records available for storage in the pre-allocated storage area of the memory.

In embodiments, the evaluation of a storage capacity of a memory for records of doses expelled with the drug delivery device may comprise a determination of the number of remaining doses for expelling with the drug delivery device by subtracting from a maximum number of dose records available for storage in a pre-allocated storage area of the memory the number of the latest dose record stored in the memory.

In embodiments, the determined data related to the approach of end of life of the drug delivery device may be evaluated and an indication of the approach of end of life of the drug delivery device may be generated based on the evaluation. Particularly, the generating of the indication may comprise generating a control signal for outputting a determined approach of end of life of the drug delivery device visible on a display and/or via a light source, audible via a sound transducer, and/or tactile via a vibrator.

In a yet further aspect the present disclosure provides a drug delivery device or a drug delivery add-on device comprising an electronic system as disclosed herein and further comprising one or more of the following: a display; a sound transducer; a vibrator; a light source; a communication interface for establishing a communication connection with an external device, wherein the electronic system is configured for transmitting determined data related to the approach of end of life of the drug delivery device to the external device for evaluation and/or outputting.

### BRIEF DESCRIPTION OF THE FIGURES

FIG. 1 shows an injection device according to an embodiment;

FIG. 2 shows a schematic block diagram of an embodiment of a device controller;

FIG. 3 shows the typical discharge behaviour of a button cell used as battery in an injection pen.

### DETAILED DESCRIPTION OF SOME EMBODIMENTS

In the following, embodiments of the present disclosure will be described with reference to injection devices, particularly an injection device in the form of a pen. The present disclosure is however not limited to such application and may equally well be deployed with other types of drug delivery devices, particularly with another shape than a pen. All absolute values are herein shown by way of example only and should not be construed as limiting.

An example of an injection pen where an injection button and grip are combined and its mechanical construction is described in detail in the international patent application WO2014033195A1. Another example of an injection device where there are separate injection button and grip components is described in WO2004078239A1.

In the following discussion, the terms “distal”, “distally” and “distal end” refer to the end of an injection pen towards which a needle is provided. The terms “proximal”, “proximally” and “proximal end” refer to the opposite end of the injection device towards which an injection button or dosage knob is provided.

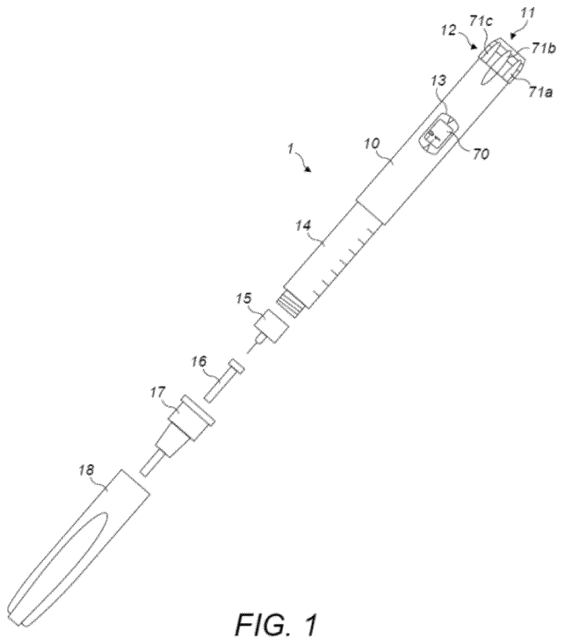

FIG. 1 is an exploded view of an injection pen 1. The injection pen 1 of FIG. 1 is a pre-filled, disposable injection pen that comprises a housing 10 and contains an insulin container 14, to which a needle 15 can be affixed. The needle is protected by an inner needle cap 16 and either an outer needle cap 17 other cap 18. An insulin dose to be ejected from injection pen 1 can be programmed, or ‘dialled in’ by turning a dosage knob 12, and a currently programmed dose is then displayed via dosage window 13, for instance in multiples of units. For example, where the injection pen 1 is configured to administer human insulin, the dosage may be displayed in so-called International Units (IU), wherein one IU is the biological equivalent of about 45.5 micrograms of pure crystalline insulin ( 1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10, which permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming. In this example, the dosage knob 12 includes one or more formations 71a, 71b, 71c to facilitate attachment of a data collection device (drug delivery or injection add-on device).

The injection pen 1 may be configured so that turning the dosage knob 12 causes a mechanical click sound to provide acoustical feedback to a user. The dial sleeve 70 mechanically inter-acts with a piston in insulin container 14. In this embodiment, the dosage knob 12 also acts as an injection button. When needle 15 is stuck into a skin portion of a patient, and then dosage knob 12 is pushed in an axial direction, the insulin dose displayed in display window 13 will be ejected from injection pen 1. When the needle 15 of injection pen 1 remains for a certain time in the skin portion after the dosage knob 12 is pushed, a high percentage of the dose is actually injected into the patient's body. Ejection of the insulin dose may also cause a mechanical click sound, which is however different from the sounds produced when rotating the dosage knob 12 during dialling of the dose.

In this embodiment, during delivery of the insulin dose, the dosage knob 12 is returned to its initial position in an axial movement, without rotation, while the dial sleeve 70 is rotated to return to its initial position, e.g. to display a dose of zero units.

Injection pen 1 may be used for several injection processes until either the insulin container 14 is empty or the expiration date of the medicament in the injection pen 1 (e.g. 28 days after the first use) is reached.

Furthermore, before using injection pen 1 for the first time, it may be necessary to perform a so-called “prime shot” to remove air from insulin container 14 and needle 15, for instance by selecting two units of insulin and pressing dosage knob 12 while holding injection pen 1 with the needle 15 upwards. For simplicity of presentation, in the following, it will be assumed that the ejected amounts substantially correspond to the injected doses, so that, for instance the amount of medicament ejected from the injection pen 1 is equal to the dose received by the user. Nevertheless, differences (e.g. losses) between the ejected amounts and the injected doses may need to be taken into account.

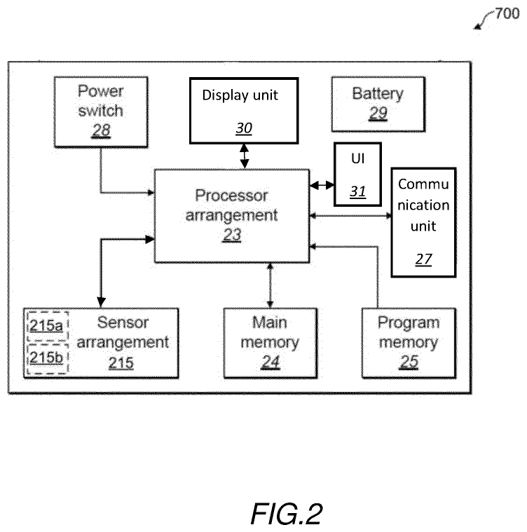

As explained above, the dosage knob 12 also functions as an injection button so that the same component is used for dialling and dispensing. A sensor arrangement 215 (FIG. 2) comprising one or more optical sensors may be mounted in the injection button or dosage knob 12 which is configured to sense the relative rotational position of the dial sleeve 70 relative to the injection button 12. This relative rotation can be equated to the size of the dose dispensed or delivered and used for the purpose of generating and storing or displaying dose history information. The sensor arrangement 215 may comprise a primary (optical) sensor 215a and a secondary (optical) sensor 215b. The sensor arrangement 215 may be also mounted in drug delivery or injection add-on device, which may be provided for usage with different injection devices 1 and configured to collect data acquired with the sensor arrangement 215.

The device 1 or an add-on device for attachment to the device 1 may also include an electronic system 700, as shown schematically in FIG. 2. The electronic system 700 may comprise the sensor arrangement 215 including the two sensors 215a, 215b and components for controlling the sensor arrangement 215 and performing other tasks such as communication with external devices, processing user inputs, outputting information for users etc. The controlling of the sensor arrangement 215 may particularly comprise a driving of at least one of the optical sensors 215a, 215b, wherein driving particularly means how to control an optical sensor to generate light pulses for measurement of a rotation of the encoder ring and to detect reflections of these measurement light pulses from reflective areas of an encoder. The electronic system may comprise a processor arrangement 23 including one or more processors, such as a microprocessor, a Digital Signal Processor (DSP), Application Specific Integrated Circuit (ASIC), Field Programmable Gate Array (FPGA) or the like, memory units 24, 25, including main memory 24 and program memory 25, which can store software for execution by the processor 20) arrangement 23, a communication unit or output 27, which may be a wireless communications interface for communicating with another device via a wireless network such as Wi-Fi™ or Bluetooth®, and/or an interface for a wired communications link, such as a socket for receiving a Universal Series Bus (USB), mini-USB or micro-USB connector, a display unit 30, for example a LCD (Liquid Crystal Display), one or more LEDs, and/or an electronic paper display, a user interface (UI) 31, for example one or more buttons and/or touch input devices, a power switch 28, and a battery 29.

The components 23, 24, 25, 27, 28, 29, 30, 31 may be soldered on a PCB containing the wiring of components. The sensor arrangement 215 may be also attached to the PCB, or may be wired with the processor arrangement 23. The implementation of the sensor unit 700 depends on the drug delivery device or drug delivery add-on device, in which it should be integrated. For example, a PCB with the components 23, 24, 25, 27, 28, 29, 30, 31 may be integrated in the distal end of the injection device 1, and the sensors 215a, 215b may be connected to the PCB via wires. At least some of the components 23, 24, 25, 27 may be also comprised by a SoC (System on Chip) or microcontroller.

A firmware stored in the program memory 25 may configure the processor arrangement 23 to control the sensor arrangement 215 such that expelling of a drug dose being delivered with the device 1 can be detected and the sensors 215a, 215b each output a sensor signal corresponding to the detected delivered drug dose. The processor arrangement 23 receives the sensor signal of each of the sensors 215a, 215b and takes readings of each sensor signal, which are processed to calculate the delivered dose. A reading may comprise for example one or more voltage samples of an analogue voltage signal of the sensor 215a, 215b. A reading may also comprise an integration of an analogue voltage signal of the sensor 215a, 215b over a certain time span. Instead of voltage signals, also electric currents, electric charges or another output signal generated by a sensor may be used for taking readings, for example frequencies of a sensor signal, frequency shifts. The readings may be taken by each sensor 215a, 215b during operation of the injection device 1 to measure the number of units dispensed by the device 1.

The firmware may further configure the processor arrangement 23 to implement functionality for determining data related to the approach of end of life of the injection pen or device 1 as will be described now in the following. Generally, four main methods for determining end of life related data can be provided, some of which also give the ability to track the approach of the end of life:
1. Battery voltage
2. Detecting device errors (related to end of life)
3. Elapsed time
4. Memory storage

These are described in more detail below with the injection pen or device 1 as shown in FIG. 1 as example. It should however be noted that the below described functionality is suitable for determining data related to end of life of any drug delivery device having at least a part of the functions of detection of device errors, storing dose records in a non-volatile memory, measuring the voltage of a device battery, connectivity functionality etc.

First, the determining of end of life related data based on the voltage of the battery 29 of the electronic system 700 is described.

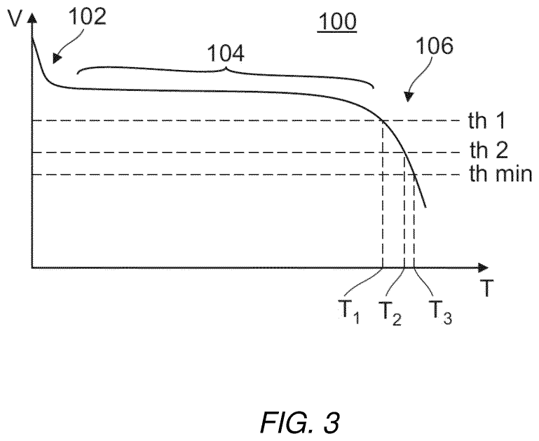

The battery 29 may be for example a primary lithium cell, with Li/MnO2 chemistry. This cell follows typical discharge behaviour 100 as shown in FIG. 3 (this discharge behaviour is similar to the one of CR1225 button cell) whereby there is a steep initial drop 102 of voltage, followed by a long plateau 104 for most of the life, followed by a relatively steep drop off 106 at the end of life.

Therefore, battery voltage alone does not facilitate easily measuring the percentage of the way through the usable life but can be used to determine end of life approach related data and particularly use the so determined data to give early warning of the approach of end of life.

Three main voltage based functions may be implemented by the firmware to determine the data. It should be noted that each of the functions can be implemented as a single functionality, or two or all three functions can be implemented together. The functions are based on measurements of the voltage of the battery 29, which can be performed by an analog-to-digital converter (ADC) of the electronic system 700 (not shown in FIG. 2). The ADC may be for example integrated in the processor arrangement 23 or comprised by the sensor arrangement 215 for converting analogue signals from the sensors 215a, 215b into digital signals. Also, a separate ADC can be provided to measure and digitize the voltage of the battery 29 for further processing by the processor arrangement 29.

A first voltage based function is based on that the voltage of the battery 29 can be measured at the end of each dose recording event and transmitted as part of a particularly extended dose record for each dose event and/or for each manual synchronisation event of the dose records with an external device via a communication link between the external device and the communication unit 27. This function may be implemented to not store the measurement of the battery voltage an internal non-volatile, for example flash memory of the injection pen 1. Every time there is a communication event via a communication link between communication unit 27 of the electronic system 700 of the pen 1 and an external device a measurement of the current battery voltage can be communicated. Therefore, the external device has the data to make its own decision and further communication of the end of life condition of the pen. Also, independent from any communication event, the processor arrangement 23 may be configured by this first function to periodically perform measurements of the current battery voltage, or a user input via the UI 31 and/or a toggling of the power switch may trigger such a measurement. The measurement of the battery voltage may also be outputted for example for displaying it on the display unit 30.

A second voltage based function is based on first threshold th1 of the battery voltage as shown in FIG. 3. The first threshold may be related to a “low voltage flag” of the injection pen 1. This second function may be particularly intended to provide the first early warning of the approach of end of life. For example, a (nominally) 3 V lithium cell in an injection pen 1 will spend most of its life between around 2.5 V and 2.7 V (under working loads) in the voltage plateau 104 after the initial drop 102. Therefore the “low voltage flag” can be set for a 3 V lithium cell used as battery 29 when the battery voltage measured at the end of a dose expelling is less than 2.4 V, about 2.4 V or greater than 2.4 V (threshold th1 in FIG. 3). The “low voltage flag” can be part of a dose record and be stored in the pen internal non-volatile memory so to be available and viewable in history, in the event that the voltage may recover slightly, or the temperature increase slightly, such that the later records are not “flagged”. The threshold th1 may be chosen to indicate that the pen 1 is leaving the plateau region 104 and will start moving towards end of life (being at the beginning of the steep drop off 106).

A third voltage based function is based on a second threshold th2 of the battery voltage as shown in FIG. 3. The second threshold may be related to a “very low voltage” error code of the injection pen 1 This third function may create an additional dose record with a special error code, which may be permanently stored and a readable event in history to indicate that the battery voltage was measured at the end of a dose to be lower than a certain threshold th2. The threshold th2 for the “very low voltage” may be chosen to seek to occur before other errors within the pen 1 that start to happen if the supply voltage starts dropping below the thresholds for which the microprocessor can run (see below). For example, in case of a 3 V lithium cell this threshold th2 may be set at about 2.3 V; this threshold may be chosen to allow for an additional instantaneous voltage drop that may occur after a dose event when the pen 1 is performing flashing of user LEDs and communications, which are higher current events and therefore pull the battery voltage lower.

The above described functions implement evaluations of the voltage of the battery 29 of the injection pen 1, which allow to determine data related to the approach of the end of life of the injection pen 1.

Second, the determining of end of life related data based on the detecting of errors of the injection pen 1 is described.

Three error based functions may be implemented by the firmware for using device errors to determine data related to the approach of end of life of the injection pen 1.

The first error based function may be based on a so-called “brown out detection”. The processor arrangement 23 may have inbuilt functionality to perform a soft reset of a microprocessor when it detects that the supply voltage is below a third threshold thmin (FIG. 3). This may be continuously detecting through the operation of the software (on a time sample basis). The third threshold thmin may be chosen to activate a so-called brown out reset for example at about 1.8 V in case of a 3 V lithium cell as battery 29. When the reset is triggered by the detection of the voltage being about 1.8 V or below the microprocessor of the processor arrangement 23 may register that the brown out event has occurred. This detection may be used to generate a device error with a specific code for brownout by the processor arrangement 23. This may further create a permanently stored and readable event in history to indicate that a brownout event has occurred. In the case of a brownout event it may represent a spontaneous reset of the software and therefore that any associated dose events may have been missed or be incorrect. Therefore, the firmware may be implemented such that the very low voltage device error, described above, is intended to occur before the brownout error.

The second error based function may be based on “power on reset detection”. This is similar to the brownout detection mechanism except this occurs when the supply voltage of the battery 29 has dropped so low that the microprocessor stops executing completely until the voltage increases again. This has the effect of “power cycling” and the microprocessor detects that it is restarting. The firmware may be implemented to assign a specific error code to this event and to create a device error record.

The third error based function may be based on “sensor health checks”. This exploits the fact that the analogue sensor readings from the sensors 215a, 215b are proportional to supply voltage of the battery 29. Therefore, as battery voltage decreases then so do the sensor readings. This has the effect that in case of optical sensors 215a, 215b the readings for reflections of these measurement light pulses from reflective areas of an encoder will decrease, as voltage decreases, and if there was no sensor health check then they could decrease so far that they are below the threshold value between black and white. However the sensor health check can detect when the final reading of the sensor 215a, 215b at the end of a dose is with 125% of this threshold. Therefore, the device error that is created in response to the sensor health check can be used as an indicator for end of life before dose record errors occur.

The above described functions implement detections of errors of the injection pen 1, which allow to determine data related to the approach of the end of life of the injection pen 1.

Elapsed time, or in-use time, can be used as an indicator for end of life. In order to create time stamps for all dose records and error records an injection pen 1 has a permanently running internal clock, a “Real Time Clock” or RTC. In order to minimise the data storage space required for time stamps each time stamp can be stored as a time offset from a “first use time”. The first time that the device 1 is activated after manufacture the device 1 may detect and store the time and date in Coordinated Universal Time as the first use time. Therefore at any point in time after this first use it is possible to determine how long the pen 1 has been operating for. The first use time and the current time can be communicated to external devices automatically and on request, for example via Bluetooth®. Therefore the external device may obtain all the information to then determine end of life predictions etc. and communicate these further to the user.

Further to the above approach there is another indicator of end of life concerned with the time. As mentioned above the time stamp for each dose can be stored as an offset from the first use time in minutes. And, in order to minimise and optimise the amount of data stored a fixed number of data bits may be assigned to this offset. The number of data bits assigned may correspond to 3.99 years of use. When this time offset is reached then a time error code can be created, stored and transmitted instead of the timestamp. Receipt of this time error code by the external device indicates an end of life condition has been reached.

Third, the determining of end of life related data based on evaluation of a storage capacity of a memory for records of doses expelled with the drug delivery device is described.

The non-volatile memory provided for storing dose records of the injection device 1 may have a limited capacity (for example, it may be implemented as a flash memory with limited storage capacity). To manage this limited storage capacity an area of memory can be pre-allocated to dose records. In such case, it may be known from the start exactly how many dose records can be created until the device will reach end of life (assuming that each dose record requires the same storage capacity).

In an injection pen there may be pre-allocated in the non-volatile dose record storage for example a capacity for 4500 dose records. At any given point in time the pen thus knows how many dose records it started with and how many are currently stored. It may make this information available to an external communicating device such that exact knowledge of the lifetime state of the device can be known or process this information with the processor arrangement 23 to determine data related to the end of life approach.

For example, the injection pen 1 may communicate via the communication unit 27 the latest dose record number as part of an extended dose record, so every automatic or manual data connection event may provide this information. In addition to this the starting dose record capacity may be available as part of the digital Unique Device Identification (UDI). The UDI may be available, on request, to an external communicating device during every communication connection, particularly Bluetooth® connection. Therefore, the external device can determine the number of doses remaining (=dose record capacity-latest dose record number-1), noting that dose record numbering may start at zero for some injection devices. The external device can similarly calculate percentage remaining or percentage used.

In this way also the latest dose record number equalling 4499 indicates that the device has reached end of life (no further record storage is possible and the pen stops functioning).

This calculation can in principle also be performed by the electronic system 700, namely the processor arrangement 23 configured accordingly by the firmware. The result can then be shown on the display unit 30 and/or transmitted to an external device via the communication unit 27, or stored in the main memory 24 or the non-volatile internal memory for later approach.

Alternative embodiments of this are possible, although not currently embodied. It would be possible to report the number of records remaining instead of the current dose record number, i.e. count down from the capacity to zero, instead of counting up.

The determined data related to the approach of end of life of the injection pen 1 can be further evaluated and an indication of the approach of end of life of the injection pen can be generated based on the evaluation. For example, it may be evaluated that the end of life of the injection pen 1 is after the next x drug expellings, this may be used to generate a corresponding indication, particularly for further processing by the processor arrangement 23 and/or for transmittal via the unit 27 to an external device. The indication may for example comprise a control signal for outputting a determined approach of end of life of the drug delivery device visible on the display unit 30 and/or via a light source, audible via a sound transducer, and/or tactile via a vibrator.

Even if the above description references a firmware to be executed by the processor arrangement 23, it should be noted that the herein disclosed functionality can also be implemented at least in part in hardware, for example as (F)PGA ((Field) Programmable Gate Array), ASIC (Application Specific Integrated Circuit).

It should also be noted that at least a part of the herein disclosed functionality can be also performed by an external computing device communicatively coupled to the drug delivery device, for example the injection pen 1. For example the measurements of the battery voltage as well as the detected errors can be transmitted to an external computing device for evaluating the received data related to the approach of end of life.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber May be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F (ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F (ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1. An electronic system (700) configured for determining data related to the approach of end of life of a drug delivery device (1) based one or more of the following:
an evaluation of the voltage of an internal battery (29) of the drug delivery device (1) or a drug delivery add-on device;
a detection of an error of the drug delivery device (1) or drug delivery add-on device, wherein the error is related to end of life of the drug delivery device (1);
an evaluation of a storage capacity of a memory (24) for records of doses expelled with the drug delivery device (1).

2. The electronic system (700) of claim 1, wherein the evaluation of the voltage of an internal battery (29) of the drug delivery device (1) or drug delivery add-on device comprises a measurement of the voltage of the internal battery (29) and an outputting of the measured voltage.

3. The electronic system (700) of claim 1 or 2, wherein the evaluation of the voltage of an internal battery (29) of the drug delivery device (1) or drug delivery add-on device comprises a measurement of the voltage of the internal battery (29) and a setting of a flag if the measured voltage is below a first threshold (th1).

4. The electronic system (700) of claim 1, 2 or 3, wherein the evaluation of the voltage of an internal battery (29) of the drug delivery device (1) or drug delivery add-on device comprises a measurement of the voltage of the internal battery (29) and a storing of an error code in a non-volatile memory (24) if the measured voltage is below a second threshold (th2).

5. The electronic system (700) of any preceding claim, wherein the detection of an error of the drug delivery device (1) or drug delivery add-on device comprises a measurement of a supply voltage of a processor (23) and a performing of a reset of the processor (23) if the measured supply voltage is below a third threshold (thmin), wherein the performed processor reset is stored in a non-volatile memory (24) as detected error.

6. The electronic system (700) of any preceding claim, wherein the detection of an error of the drug delivery device (1) or drug delivery add-on device comprises a measurement of a supply voltage of a processor (23) and a stopping of operation of the processor (23) as long as the measured supply voltage is below a fourth threshold, wherein the processor operation stopping is stored in a non-volatile memory (24) as detected error.

7. The electronic system (700) of any preceding claim, wherein the detection of an error of the drug delivery device (1) or drug delivery add-on device comprises an evaluation of one or more readings of a sensor (215a, 215b) of the drug delivery device (1) or drug delivery add-on device and a detection of an error if the evaluation of the one or more readings indicates a low supply voltage.

8. The electronic system (700) of any preceding claim, wherein the evaluation of a storage capacity of a memory (24) for records of doses expelled with the drug delivery device (1) comprises a determination of the number of dose records expelled with the drug delivery device (1) and currently stored in a pre-allocated storage area of the memory (24), and a determination of the remaining storage capacity based on the determined number and a maximum number of dose records available for storage in the pre-allocated storage area of the memory (24).

9. The electronic system (700) of any preceding claim, wherein the evaluation of a storage capacity of a memory (24) for records of doses expelled with the drug delivery device (1) comprises a determination of the number of remaining doses for expelling with the drug delivery device (1) by subtracting from a maximum number of dose records available for storage in a pre-allocated storage area of the memory (24) the number of the latest dose record stored in the memory (24).

10. The electronic system (700) of any preceding claim being further configured for evaluating the determined data related to the approach of end of life of the drug delivery device (1) and generating an indication of the approach of end of life of the drug delivery device (1) based on the evaluation, wherein the indication particularly comprises a control signal for outputting a determined approach of end of life of the drug delivery device (1) visible on a display (30) and/or via a light source, audible via a sound transducer, and/or tactile via a vibrator.

11. A computer-implemented method for determining data related to the approach of end of life of a drug delivery device (1) based one or more of the following:
evaluating the voltage of an internal battery (29) of the drug delivery device (1) or a drug delivery add-on device;
detecting an error of the drug delivery device (1) or drug delivery add-on device, wherein the error is related to end of life of the drug delivery device (1);
evaluating a storage capacity of a memory (24) for records of doses expelled with the drug delivery device (1).

12. The method of claim 11, wherein the evaluating of the voltage of an internal battery (29) of the drug delivery device (1) or drug delivery add-on device comprises one or more of the following:
measuring the voltage of the internal battery (29) and outputting of the measured voltage;
measuring the voltage of the internal battery (29) and setting of a flag if the measured voltage is below a first threshold;
measuring the voltage of the internal battery (29) and storing of an error code in a non-volatile memory (24) if the measured voltage is below a second threshold.

13. The method of claim 11 or 12, wherein the detecting of an error of the drug delivery device (1) or drug delivery add-on device comprises one or more of the following:
measuring a supply voltage of a processor (23) and performing of a reset of the processor (23) if the measured supply voltage is below a third threshold (thmin), wherein the performed processor reset is stored in a non-volatile memory (24) as detected error;
measuring a supply voltage of a processor (23) and stopping of operation of the processor (23) as long as the measured supply voltage is below a fourth threshold, wherein the processor operation stopping is stored in a non-volatile memory (24) as detected error;
evaluating one or more readings of a sensor (215a, 215b) of the drug delivery device (1) or drug delivery add-on device and a detection of an error if the evaluation of the one or more readings indicates a low supply voltage.

14. The method of claim 11, 12 or 13, wherein the evaluating of a storage capacity of a memory (24) for records of doses expelled with the drug delivery device (1) comprises one or more of the following:
determining the number of dose records expelled with the drug delivery device (1) and currently stored in a pre-allocated storage area of the memory (24), and determining the remaining storage capacity based on the determined number and a maximum number of dose records available for storage in the pre-allocated storage area of the memory (24);
determining the number of remaining doses for expelling with the drug delivery device (1) by subtracting from a maximum number of dose records available for storage in a pre-allocated storage area of the memory (24) the number of the latest dose record stored in the memory (24).

15. The method of any of claims 11 to 14 further comprising evaluating the determined data related to the approach of end of life of the drug delivery device (1) and generating an indication of the approach of end of life of the drug delivery device (1) based on the evaluation, wherein the generating of the indication particularly comprises generating a control signal for outputting a determined approach of end of life of the drug delivery device (1) visible on a display (30) and/or via a light source, audible via a sound transducer, and/or tactile via a vibrator.
